(19) United States
(12) Patent Application Publication
(10) Pub. No.: US 2006/0159770 A1
(43) Pub. Date: Jul. 20, 2006

(54) Composition for treating and/or preventing dysfunctions associated with type 2 diabetes mellitus

(76) Inventors: Clara Lucia Garcia-Rodenas, Residence Le Frene (CH); Martial Dangin, Avenue du Stade (FR); Paolo Tessari, Via Belzoni (IT); John Bruce, Residence la Bergerie (CH)

Correspondence Address: BELL, BOYD & LLOYD LLC, P. O. BOX 1135, CHICAGO, IL, 60690-1135, US

(21) Appl. No.: 10/562,842

(22) PCT Filed: Jun. 30, 2004
(86) PCT No.: PCT/EP04/07094

§ 371 Date: Dec. 29, 2005

(30) Foreign Application Priority Data
Jun. 30, 2003 (EP) 03014816.7

(51) Int. Cl. A61K 35/20

(52) U.S. Cl. 424/535

(57) ABSTRACT

The present invention relates to the use of a composition for treating, preventing and/or improving metabolic dysfunctions associated with Type 2 diabetes mellitus and insulin resistance, said composition comprising intact whey protein, and to nutritional or pharmaceutical compositions and functional food products.

The present invention relates to the treatment and/or prevention of dysfunctions associated with Type 2 diabetes mellitus and/or insulin resistance.

Diabetes mellitus and insulin resistance both are metabolic disorders exhibiting a major common manifestation, hyperglycaemia.

Diabetes mellitus originates from an inherited and/or acquired deficiency in the production of insulin by the pancreas, or by the ineffectiveness of the insulin produced. Such a deficiency eventually results in increased concentrations of glucose in the blood, which in turn damage many of the body's systems, in particular the blood vessels and nerves.

There are two principle forms of diabetes, Type 1 and Type 2.

In Type 1 diabetes the pancreas of affected individuals fails to produce insulin largely due to a destruction of the islets of Langerhans, which in most cases seem to occur as a consequence of an auto-immune reaction triggered by some environmental factor, such as a viral infection. Heavy lymphocytic infiltrates appear in and around islets with the number and size of islets being reduced, eventually leading to decreased insulin production and glucose intolerance. This form develops most frequently in children and adolescents, but is being increasingly noted later in life.

Type 2 diabetes results from the body's inability to properly respond to the action of insulin produced by the pancreas. It occurs most frequently in adults, but is being noted increasingly in adolescents as well. The islets of Langerhans are normal in number or somewhat reduced with type II diabetes mellitus. Fibrosis and deposition of amylin polypeptide within islets are most characteristic of the chronic states of Type 2 diabetes.

Diabetes mellitus of both types is associated with a number of life-threatening and/or handicapping diseases. Examples are nodular and diffuse glomerulosclerosis, which may lead to chronic renal failure. Diabetics are prone to infections, particularly pyelonephritis. Also the eyes may be affected with diabetic retinopathy being one of the leading causes for irreversible blindness. Most persons with Type 1 diabetes and many of those with Type 2 diabetes develop some sort of background (non-proliferative) retinopathy. In severe cases, neo-vascularization may lead to adhesions (synechiae) between iris and cornea or iris and lens, eventually leading to secondary glaucoma with blindness. Also cataracts are more common in diabetics. This predilection for development of cataracts is felt to result from hyperglycaemia leading to accumulation of sorbitol that results in osmotic damage to the crystalline lens.

Persons with diabetes mellitus, either Type 1 or Type 2, also exhibit early and accelerated atherosclerosis. The most serious complications of this are atherosclerotic heart disease, cerebrovascular disease, and renal disease, with the most common cause of death being myocardial infarction. Peripheral vascular disease is a particular problem with diabetes mellitus and is made worse through the development of diabetic neuropathy, leading to propensity for injury. Mucormycosis is another feared complication in individuals experiencing diabetes mellitus. The site of involvement is typically the nasopharyngeal region, but the infection can spread to involve soft tissues and bone of the face, orbit, skull, and brain.

The treatment of individuals suffering from diabetes generally involves physical activity, diet and/or administration of medicaments. People with Type 1 diabetes are usually totally dependent on insulin injections for survival, requiring daily administration. Type 2 diabetic patients usually have to observe a strict diet and may additionally receive oral anti-diabetics, such as sulphonyl ureas, alpha-glucosidase inhibitors and biguanides, or even injections of insulin, the administration of which is often associated with severe side effects and complications.

The majority of people suffer from Type 2 diabetes, which accounts for around 90% of all diabetes cases world-wide. On the molecular level Type 2 diabetes is characterized by a defect of both, insulin secretion and action. The defect of insulin secretion relates mostly to the first phase of the post-prandial insulin release from pancreas, wherein in diabetic patients the already formed insulin is stored within the β-cells, but cannot be released into circulation. Indeed, most of the Type 2 diabetic patients present a resistance to the action of the insulin such that in order to cope with similar glucose concentration as present in healthy people, Type 2 diabetics require a higher concentration of insulin in plasma.

Another type of abnormalities in glucose metabolism is insulin resistance, that is, a reduced sensitivity in the tissues of the body to the action of insulin, which goes along with a perturbed lipid (blood fats) metabolism, obesity, and high blood pressure. This cluster of abnormalities has come to be known as a syndrome, going by a variety of names, including Syndrome X, the Deadly Quartet, and the Insulin Resistance Syndrome.

When insulin resistance, or reduced insulin sensitivity, exists, the body attempts to overcome this resistance by secreting more insulin from the pancreas. The development of Type 2, or non-insulin dependent, diabetes occurs when the pancreas fails to sustain this increased insulin secretion. The importance of the Insulin Resistance Syndrome, or perhaps more accurately, “The Pluri-Metabolic Syndrome”, lies in its consequences. The syndrome is typically characterized by varying degrees of glucose intolerance, abnormal cholesterol and/or triglyceride levels, high blood pressure, and upper body obesity, all independent risk factors for cardiac disease.

Following a meal, a person suffering insulin resistance will have elevated glucose circulating in the blood, signalling yet more insulin to be released from the pancreas until the glucose is taken up by the cells. Experts suggest that 11 to 25 percent of the adult population may be resistant to insulin to some degree.

The use of milk protein hydrolysates to improve glucose metabolism or control glycaemic response in individuals suffering from diabetes has already been suggested in US Patent Application No. 2003/0004095. According to this document, milk protein hydrolysates, specifically caseinoglycomacropeptide can induce the release of GLP-1, a peptide known to be capable of potentiating glucose-induced insulin secretion as well as stimulating proinsulin gene expression and proinsulin biosynthesis. However, milk protein hydrolysates are known to have an unpleasant bitter taste which may adversely affect patient compliance with a regime based on these products.

Due to the increasing number of affected people world-wide and the changing lifestyle of the society there exists a need in the art to provide additional means useful in preventing, treating and/or improving conditions associated with Type 2 diabetes mellitus and/or insulin resistance. Moreover, such a means should be essentially free from disadvantageous side-effects well known from many oral anti-diabetics, and should be easy to take up.

The present invention provides a method of treating, preventing and/or improving metabolic dysfunctions and conditions associated with Type 2 diabetes mellitus by administering an effective amount of a composition containing intact whey proteins

The inventors have found that intact whey proteins induce a dramatic but short increase in plasma amino acids as opposed to proteins such as casein which are more slowly digested and which induce a mild but prolonged plateau of hyperaminoacidemia (Boirie et al, Proc. Natl. Acad. Sci. USA, 1997, 94:14930-5).

Intact whey protein may be present in the composition in an amount of from about 1 to 90% by weight, preferably from 5 to 70% by weight, more preferably 11 to 60% by weight, even more preferably 21 to 40% by weight and most preferably about 25 to 35% by weight, on the basis of the total dry weight of the composition. Preferable the whey protein is sweet whey protein.

It has been found that intact whey proteins have a particular effect after consumption by type 2 diabetes patients. According to the studies carried out intact whey proteins significantly increase the production and/or secretion of insulin, as determined by an increase in the maximal plasma concentration and bio-availability of pro-insulin, insulin and C-peptide. The C-peptide results from the formation of biological active insulin from pro-insulin and serves as an indicator showing how much insulin is produced in an individual. C-peptide is considered to represent the most accurate indicator for the production of insulin in β-cells. In other words, intact whey proteins enhance post-prandial insulinemia and help to restore the first phase of the insulin response of diabetic patients to a standard meal and the kinetics of post-prandial insulinemia provoked by dietary carbohydrates may thus be accurately modulated by such proteins.

The present composition will also be of high interest for large parts of the population, which are not suffering from insulin resistance or Type 2 diabetes mellitus at present, but belong to a target group at risk to develop any of said disorders, either due to a high risk diet or genetic predisposition. Moreover, an enhancement of post-prandial insulinemia is also highly interesting for other groups of persons, such as patients recovering from diseases or trauma leading to muscle depletion, exercising persons or elderly persons, since insulin is an anabolic hormone necessary for muscle mass maintenance and growth. High post-prandial insulinemia therefore promotes improving muscle mass accretion in exercising persons, is helpful for patients suffering from muscle depletion, and supports muscle maintenance in elderly persons.

The composition as described above may of course also be used for the manufacture of a so called functional food product or a pharmaceutical composition.

Particularly good results may be achieved when providing at least 0.1 g intact whey proteins per kg body weight, more preferably 0.5 to 0.8 g intact whey proteins per kg body weight, e.g. during, before or after a standard meal, in particular a standard meal comprising carbohydrates. A standard meal is any meal comprising at least 150 kcal, more preferably at least 250 kcal.

The composition is preferably enterally administrable, such as in form of a powder, a liquid concentrate, or a ready-to-drink beverage. The composition can be directly consumed or admixed with various foodstuffs, in particular to ready-to-use snacks, dairy products or drinks, or used for the preparation of an oral or enteral nutritional composition or a fruit juice.

The composition according to the present invention may of course comprise other conventional ingredients, such as vitamins and minerals, dietary fibres, fat, food additives etc..

In particular, vitamins and minerals may be present in an amount of between 30% and 150% of US RDA (US recommended (daily) dietary allowance) per daily dosage. Additionally, one or more food grade emulsifiers may be included in the nutritional composition, if desired, such as diacetyl tartaric acid esters of mono- and diglycerides, lecithin, and mono- or diglycerides or a mixture thereof. Similarly, suitable food-acceptable salts and/or stabilizers may also be included.

If desired, fibres either soluble and insoluble may be included.

If a lipid source is included, it preferably comprises about 5% to 40% of the energy (measured in calories) on the basis of the total energy of the composition; preferably, about 10% to about 20% of the energy. Any suitable fat or fat mixture may be used. Vegetable fat is particularly suitable, for example soy oil, palm oil, coconut oil, safflower oil, sunflower oil, corn oil, canola oil, lecithin and the like. Animal fat such as milk fat may also be added if desired.

If a carbohydrate source is included, it preferably comprises less than 10% by weight, preferably less than 5% by weight, more preferably less than 1% by weight of the composition. For some applications, such as e.g. ready-to-use beverages, compositions are advantageous which are essentially free from, or comprise less than 5% by weight of, mono-saccharides. If monosaccharides are present, glucose galactose and tagatose each preferably account for less than 40% by weight, more preferably less than 10% by weight, even more preferably less than 1% by weight of the mono-saccharides. In other applications such as ready-to-use snacks, however, inclusion of a carbohydrate source may be advantageous, preferably in an amount to provide 1 to 70%, more preferably 25 % to 45% of the energy on basis of the total energy of the composition.

Non-caloric sweeteners, flavourings and food-acceptable colourings may also be included.

A particularly advantageous embodiment comprises a liquid composition such as a ready-to-use beverage based on fruit juice, vegetable juice, water, isotonic drinks, carbonated flavoured drinks, soft drinks, teas, coffees, dairy products, meat and/or vegetable soups or mixtures thereof, which may be supplemented with minerals, vitamins and/or carbonic acid, if desired. Beverages comprising fruit or vegetable juices provide additionally the advantage of comprising vitamins, minerals or even enzymes and provide an advantageous complementation of a nutritional composition according to the present invention. In particular, juices such as orange, apple, pineapple, grapefruit, lemon, lime, mango, passion fruit, elderberries, cranberries, currants, grape, tomato, carrot or combinations thereof may form the basis for a ready-to-use beverage.

A liquid composition may comprise from 11 to 97% by weight, preferably from 21 to 80% by weight, most preferably from 61 to 75% by weight, of any of the before-mentioned juices, beverages, water or mixtures thereof, and from 3 to 89% by weight, preferably from 20 to 79% by weight, most preferably from 25 to 39% by weight, of a composition according to the present invention, on basis of the total weight of the liquid composition.

A liquid composition will preferably include 1 to 20% by weight intact whey proteins, more preferably 5 to 12% by weight intact whey proteins.

Advantageously, a beverage according to the present invention delivers 1 to 150 kcal, preferably 21 to 100 kcal, more preferably 31 to 50 kcal per 100 g of fluid preparation. For example, a beverage accompanying a standard meal may e.g. provide per dosage (i.e. per standard meal) 40 to 60 g of intact whey proteins.

Of course, consumers may also prepare such a beverage by mixing a composition according to the present invention (e.g. according to instructions on the package) with a beverage of their choice.

Alternatively, a food product may be enriched with a composition according to the present invention. For example, a fermented milk, a yoghurt, a fresh cheese, a renneted milk, a confectionery bar, breakfast cereal flakes or bars, a drink, milk powder, soy-based product, non-milk fermented product or a nutritional supplement for clinical nutrition. Then, the amount of the composition added is preferably, at least 0.5% by weight, more preferably 11 to 40% by weight, on basis of the total weight of the food product.

Food products or beverages as detailed above, provide the advantage that they may be consumed shortly before, during, or shortly after a meal by a person, in particular from a person suffering from Type 2 diabetes, and permit an easy solution for enhancing post-prandial insulinemia. Thus, compositions according to the present invention may be helpful in significantly increasing the quality of life of large groups of the population.

A composition according to the present invention may also be used for the preparation of an enteral nutritional formula, in particular for patients suffering from muscle depletion or for supporting muscle maintenance.

Compositions according to the present invention may be designed both for human consumption and for consumption by a companion animal, in particular for dogs and cats.

All before-mentioned products according to the present invention provide the advantage that they may be expected to be highly accepted by the consumers as they are formulated on basis of well-known nutritional components,. which proved to be essentially free of undesired side-effects. Moreover, compositions according to the present invention are essentially free of unpleasant tastes and may be regularly, e.g. daily consumed.

According to another aspect, the invention also provides a method for treating or preventing metabolic dysfunctions and/or improving conditions associated with Type 2 diabetes mellitus or insulin resistance which comprises administering an effective amount intact whey proteins.

The following examples are given by way of illustration only and should not be construed as limiting the subject-matter of the present application.

### EXAMPLE 1

Influence of Protein on the Insulin Response

This example demonstrates the effect of the protein digestion rate and compares the effects of intact whey and casein proteins.

The following abbreviations are used:

Cₘₐₓ is the maximal plasma concentration of a compound, Tₘₐₓ is time to achieve Cₘₐₓ, AUC is the area under the plasma concentration curve versus time, and p is the treatment effect.

The following regimens were administered:
Treatment with sweet whey protein isolate (Lacprodan DI-9224, Arla Foods, Denmark), a protein which is providing for a rapid digestion rate (labelled with code “S1” below);
Treatment with micellar casein (Promilk 852B, Ingredia Lactoprosperité AG, Switzerland), a comparative protein providing an essentially slow protein digestion rate (labelled with code “S2” below);

Each regimen was a protein powder and was reconstituted in a liquid form by mixing 100 g of protein powder with 900 g water. This solution delivers 40 kcal and 10 g protein per 100 g. Formula dosage depends on patient weight: 7 g liquid formula/kg body weight (BW) (0.7 g protein/kg BW). It was administrated as part of a test meal (6 kcal/kg BW; 38% carbohydrates, 15% lipids and 47% proteins). In order to obtain a high insulin response allowing for product discrimination, the protein solutions were ingested with carbohydrates and lipids (bread and chocolate spread) and the amount of protein was relatively high (around ⅔ of the protein daily requirements).

Study Setup

The study was designed as a double-blind, single center, exploratory, randomized and controlled cross-over clinical trial. It has been carried out at the Centro Antidiabetico, Azienda Ospedaliera de Padova, Italy. The subjects were Type 2 diabetic patients. The treatments were blind to patients and to the study staff. Patients received once each treatment with a wash-out period of at least 2 weeks between treatments.

The study was performed on Type 2 diabetic patients of both sexes aged between 31 to 65 years from the outpatient diabetic subjects scheduled to be regularly visited at the Centro Antidiabetico of the Azienda Ospedialera of Padova.

The inclusion criteria were: more than 3 years of disease; defective endogenous insulin secretion [C-peptide response peak after iv glucagon≦3 mg/ml]; age: 30-65 years; 18<BMI (Body Mass Index)<30 kg /m²; having obtained his/her informed consent; diet and/or OHA (oral hypoglycaemic agent)-treated.

The exclusion criteria were: treated with insulin; patients with moderate to severe kidney or liver insufficiency, respiratory or cardiac failure, endocrinopathies other than diabetes, and major diseases of the GI tract causing malabsorption; patients who cannot be expected to comply with the treatment; currently participating or having participated in another clinical trial during the last 3 months prior to the beginning of this study.

Each subject consumed both test treatments once in random order. The test period lasted one day. Twenty-four hours before and during test day, the patients had to interrupt the OHA therapy. At test day, the patients came to the hospital after overnight fasting. After placing an indwelling catheter in the patient arm, and taking two basal blood samples, patients consumed the test meal. The test meal included a liquid formula containing one of the treatments. Blood sampling was done at −10, 0, 10, 20, 30, 60, 90, 120, 150 and 180 minutes of the test meal intake.

Data Collection, Management and Validation

The following data were collected:

Test Periods
Blood parameters: for each test period, the blood was collected for 190 minutes (2 samples before and 8 samples after the test meal). Then, the following plasmatic parameters were measured:
   amino acids: taurine, aspartate, threonine, serine, asparagine, glutamate, glutamine, proline, glycine, alanine, citrulline, valine, cystine, methionine, isoleucine, leucine, tyrosine, phenylalanine, tryptophan, ornithine, lysine, histidine and arginine.
   hormones and metabolites: proinsulin, insulin, GLP-1, GIP, glucagon, glucose, C-peptide, triglycerides and total cholesterol.
Anthropometric measures: weight and height immediately before the test meal.

Statistical Methods

Statistical Analyses Planned in the Protocol

The primary and secondary outcomes were analyzed by using a linear mixed-effect model with the two treatments and sex as fixed effects and subject as random effect. The results include: mean±standard deviation and 95% confidence interval for mean difference. The rejection level in statistical tests was equal to 5% (p=0.05). The statistical analyses were done using SAS software (version 8.2).

Calculations of the Kinetic Parameters

AUC is the area under the plasma concentration curve versus time. It is calculated by the trapezoidal rule as follows:

$$AUC = \frac{1}{2} {\sum }_{i = 1}^{n - 1} ( {T}_{i + 1} - {T}_{i} ) ( {C}_{i + 1} + {C}_{i} - 2 B )$$

where Tᵢ is the i^{th} time value, Cᵢ is the i^{th} concentration value, n is the number of time values and B is the baseline value. The kinetic parameters were calculated using NCSS2000 software.

Results

Compliance

For each test meal, the average amounts of bread, chocolate spread and protein drink are 27.46±3.97 g of bread, 20.85±3.00 g of chocolate and 487±70 g of protein drink.

Primary Outcome: Plasmatic C-peptide

The kinetic parameters of C-peptide [AUC_{(0-180 min)}, Cₘₐₓ and Tₘₐₓ) from the treatments are summarized in Tables 2.

TABLE 1-a
AUC_{(0-180 min)} of C-peptide [(ng/ml) * min]

| | |
|---|---|
| DESCRIPTIVE | TREATMENT |

| STATISTICS | “Whey protein” S1 | “Casein” S2 |
|---|---|---|
| N | 12 | 11 |
| Mean | 672 | 572 |
| ±SD | 268 | 213 |
| 95% CI | [502; 843] | [428; 715] |
| Minimum | 385 | 291 |
| Median | 570 | 552 |
| Maximum | 1213 | 901 |

| | |
|---|---|
| [S1 − S2] | 122 ± 82 (SE = 35) |
| | [50: 193] |

Mean ± standard deviation;
[ ]: 95% confidence interval for mean;
SE: Standard error of the mean;
SD (standard deviation);
CI (confidence interval).

The bioavailability [AUC_{(0-180 min)}] is significantly different between the treatments. The AUC of treatment [S2; “casein”] is significantly lower than [S1; “whey protein”] (p=0.002).

TABLE 1-b
Cₘₐₓ of C-peptide [ng/ml]

| | |
|---|---|
| DESCRIPTIVE | TREATMENT |

| STATISTICS | “Whey protein” S1 | “Casein” S2 |
|---|---|---|
| N | 12 | 11 |
| Mean | 5.11 | 4.33 |
| ±SD | 2.23 | 1.47 |
| 95% CI | [3.70; 6.52] | [3.34; 5.32] |
| Minimum | 2.76 | 1.88 |
| Median | 4.90 | 4.36 |
| Maximum | 10.40 | 6.20 |

| | |
|---|---|
| [S1 − S2] | 0.94 ± 0.84 (SE = 0.35) |
| | [0.21: 1.67] |

Mean ± standard deviation;
[ ]: 95% confidence interval for mean;
SE: Standard error of the mean;
SD (standard deviation);
CI (confidence interval).

Cₘₐₓ, i.e. the maximal plasma concentration of the C-peptide, is significantly different between the treatments. The Cₘₐₓ of treatment [S2; “casein”] is significantly lower than [S1; “whey protein”] (p=0.015).

TABLE 1-c
Tₘₐₓ to reach the Cₘₐₓ of C-peptide [min.]

| | |
|---|---|
| DESCRIPTIVE | TREATMENT |

| STATISTICS | “Whey protein” S1 | “Casein” S2 |
|---|---|---|
| N | 12 | 11 |
| Mean | 117 | 129 |
| ±SD | 24 | 54 |
| 95% CI | [102; 132] | [93; 165] |
| Minimum | 80 | 15 |
| Median | 127 | 138 |
| Maximum | 145 | 206 |

| | |
|---|---|
| [S1 − S2] | −10 ± 34 (SE = 14) |
| | [−40: 19] |

Mean ± standard deviation;
[ ]: 95% confidence interval for mean;
SE: Standard error of the mean;
SD (standard deviation);
CI (confidence interval).

Tₘₐₓ, i.e. the time to reach the maximal plasma concentration of the C-peptide Cₘₐₓ is not significantly different between the treatments (p=0.43).

Secondary Outcomes: Hormones and Metabolites

Proinsulin

Proinsulin is a precursor of insulin, which is obtained after cleavage of the C-peptide and after formation of S-S-bridges. The kinetic parameters of proinsulin (AUC_{(0-180 min)}, Cₘₐₓ and Tₘₐₓ) for the treatments are summarized in Table 2.

TABLE 2
Kinetic parameters of proinsulin

| KINETIC | DIFFERENCE BETWEEN THE | TREATMENT |
|---|---|---|
| PARAMETERS | TREATMENTS [S1 − S2] | EFFECT |
| AUC [(pM) * min] | 752 ± 1189 | p > 0.05 |
| | [−329; 1834] | |
| Cₘₐₓ (pM) | 8 ± 7 | p > 0.05 |
| | [1; 15] | |
| Tₘₐₓ (min) | −26 ± 26 | p > 0.05 |
| | [−49; −3] | |

Mean ± standard deviation;
[ ]: 95% confidence interval for mean difference.

Between 1 to 180 minutes, the plasmatic amount of proinsulin (bioavailability) and Cₘₐₓ of proinsulin with the treatment [S2; “casein”] does not significantly differ from [S1; “whey protein”]. Tₘₐₓ of proinsulin is not significantly different between the treatments (p=0.07).

Insulin

The kinetic parameters of insulin (AUC_{(0-180 min)}, Cₘₐₓ and Tₘₐₓ) from the treatments are summarized in Table 3.

TABLE 3
Kinetic parameters of insulin

| KINETIC | DIFFERENCE BETWEEN THE | TREATMENT |
|---|---|---|
| PARAMETERS | TREATMENTS [S1 − S2] | EFFECT |
| AUC [(μU/ml) * min] | 1052 ± 1139 | p > 0.05 |
| | [56; 2049] | |
| Cₘₐₓ (μU/ml) | 13 ± 12 | p = 0.014 |
| | [3; 24] | |
| Tₘₐₓ (min) | −7 ± 37 | p > 0.05 |
| | [−40; 25] | |

Mean ± standard deviation;
[ ]: 95% confidence interval for mean difference.

Between 0 and 180 minutes, the plasmatic amount of insulin (bioavailability) with the treatment [S2; “casein”] is not significantly different from [S1; “whey protein”]. Cₘₐₓ of insulin with the treatment [S2; “casein”] is significantly lower than with the treatment [S1; “whey protein”] (p=0.014). Tₘₐₓ of insulin is not significantly different between the treatments (p=0.86).

Glucagon

Glucagon is a polypeptide hormone formed in the pancreas. The kinetic parameters of glucagon (AUC_{(0-180 min)}, Cₘₐₓ and Tₘₐₓ) from the treatments are summarized in Table 4.

TABLE 4
Kinetic parameters of glucagon

| KINETIC | DIFFERENCE BETWEEN THE | TREATMENT |
|---|---|---|
| PARAMETERS | TREATMENTS [S1 − S2] | EFFECT |
| AUC [(μg/L) * min] | 3955 ± 6311 | p > 0.05 |
| | [−1473; 9382] | |
| Cₘₐₓ (μg/L) | 78 ± 84 | p > 0.05 |
| | [4; 152] | |
| Tₘₐₓ (min) | 15 ± 24 | p > 0.05 |
| | [−5; 36] | |

Mean ± standard deviation;
[ ]: 95% confidence interval for mean difference.

Between 0 and 180 minutes, the plasmatic amount of glucagon (bioavailability) with the treatments [S1] and [S2] is not significantly different (p=0.13). Cₘₐₓ of glucagon with the treatments [S1] and [S2] is not significantly different (P=0.10). Tₘₐₓ of glucagonis not significantly different between the treatments (p=0.29).

As becomes obvious from the results above, the treatment with whey protein does not have a significant influence with respect to glucagon, a hormone which could give rise to an undesired blood sugar increase.

Glucose

The kinetic parameters of glucose (AUC_{(0-180 min)}, Cₘₐₓ and Tₘₐₓ) from the treatments are summarized in Table 5.

TABLE 5
Kinetic parameters of glucose

| KINETIC | DIFFERENCE BETWEEN THE | TREATMENT |
|---|---|---|
| PARAMETERS | TREATMENTS [S1 − S2] | EFFECT |
| AUC [(mg/dl) * min] | −1447 ± 3533 | p > 0.05 |
| | [−4487; 1592] | |
| Cₘₐₓ (mg/dl) | −7 ± 18 | p > 0.05 |
| | [−23; 9] | |
| Tₘₐₓ (min) | 1 ± 31 | p > 0.05 |
| | [−26; 29] | |

Mean ± standard deviation;
[ ]: 95% confidence interval for mean difference.

Between 0 and 180 minutes, neither the plasmatic amount of glucose (bioavailability) nor the Cₘₐₓ of glucose nor the Tₘₐₓ of glucose is significantly different between the treatments (p=0.66).

GIP (Gastric Inhibitory Peptide)

GIP (Gastric Inhibitory Peptide) is a gastrointestinal hormone which inhibits the liberation of insulin. The kinetic parameters of GIP (AUC_{(0-150 min)}, Cₘₐₓ and Tₘₐₓ) from the three treatments are summarized in Table 6.

TABLE 6
Kinetic parameters of GIP

| KINETIC | DIFFERENCE BETWEEN THE | TREATMENT |
|---|---|---|
| PARAMETERS | TREATMENTS [S1 − S2] | EFFECT |
| AUC [(pM) * min] | 343 ± 1260 | p > 0.05 |
| | [−442; 1128] | |
| Cₘₐₓ (pM) | 0 ± 15 | p > 0.05 |
| | [−10; 9] | |
| Tₘₐₓ (min) | −10 ± 52 | p > 0.05 |
| | [−42; 22] | |

Mean ± standard deviation;
[ ]: 95% confidence interval for mean difference.

Between 0 and 150 minutes, neither the plasmatic amount of GIP (bioavailability) nor the Cₘₐₓ of GIP nor the Tₘₐₓ of GIP is significantly different between the treatments (p=0.61).

GLP-1 (Glucagon Like Polypeptide)

GLP-1 is a gastrointestinal hormone that has been reported to stimulate the release of insulin by the pancreatic beta-cells.

The kinetic parameters of GLP-1 (AUC_{(0-150 min)}, Cₘₐₓ and Tₘₐₓ) from the treatments are summarized in Table 7

TABLE 7
Kinetic parameters of GLP-1

| KINETIC | DIFFERENCE BETWEEN THE | TREATMENT |
|---|---|---|
| PARAMETERS | TREATMENTS [S1 − S2] | EFFECT |
| AUC [(pM) * min] | 234 ± 399 | p = 0.05 |
| | [−23; 490] | |
| Cₘₐₓ (pM) | 4 ± 8 | p > 0.05 |
| | [−1; 8] | |
| Tₘₐₓ (min) | 4 ± 43 | p > 0.05 |
| | [−23; 32] | |

Mean ± standard deviation;
[ ]: 95% confidence interval for mean difference.

Between 0 and 150 minutes, the plasmatic amount of GLP-1 (bioavailability) is not significantly different with the treatments [S1] and [S2] (p=0.05). Cₘₐₓ of GLP-1 is not significantly different between the treatments (p=0.09). Tₘₐₓ of GLP-1 is not significantly different between the treatments (p=0.32).

Total Cholesterol

The kinetic parameters of total cholesterol (AUC_{(0-180 min)}, Cₘₐₓ and Tₘₐₓ) from the treatments are summarized in Table 8.

TABLE 8
Kinetic parameters of total cholesterol

| KINETIC | DIFFERENCE BETWEEN THE | TREATMENT |
|---|---|---|
| PARAMETERS | TREATMENTS [S1 − S2] | EFFECT |
| AUC [(mg/dl) * min] | −1273 ± 3318 | p > 0.05 |
| | [−4127; 1581] | |
| Cₘₐₓ (mg/dl) | −6.70 ± 18 | p > 0.05 |
| | [−22; 9] | |
| Tₘₐₓ (min) | 11 ± 42 | p > 0.05 |
| | [−26; 47] | |

Mean ± standard deviation;
[ ]: 95% confidence interval for mean difference.

Between 0 and 180 minutes, the plasmatic amount of total cholesterol (bioavailability) is not significantly different between the treatments [S1] and [S2] (p=0.62). Cₘₐₓ of cholesterol is not significantly different between the treatments [S1] and [S2] (p=0.59). Tₘₐₓ of cholesterol is not significantly different between the treatments (p=0.33).

This example shows that administration of intact whey proteins is not associated with a negative effect with respect to the cholesterol level in blood.

Triglycerides

The kinetic parameters of triglycerides (AUC_{(0-180 min)}, Cₘₐₓ and Tₘₐₓ) from the treatments are summarized in Table 9.

TABLE 9
Kinetic parameters of triglycerides

| KINETIC | DIFFERENCE BETWEEN THE | TREATMENT |
|---|---|---|
| PARAMETERS | TREATMENTS [S1 − S2] | EFFECT |
| AUC [(mg/dl) * min] | −454 ± 3727 | p > 0.05 |
| | [−3660; 2753] | |
| Cₘₐₓ (mg/dl) | −13 ± 27 | p > 0.05 |
| | [−37; 11] | |
| Tₘₐₓ (min) | −19 ± 53 | p > 0.05 |
| | [−65; 27] | |

Mean ± standard deviation;
[ ]: 95% confidence interval for mean difference.

Between 0 and 180 minutes, the plasmatic amount of triglycerides (bioavailability) is not significantly different between the treatments [S1] and [S2] (p=0.96). Cₘₐₓ of triglycerides is not significantly different with the treatments [S1] and [S2] (p=0.36). Tₘₐₓ of triglycerides is not significantly different between the treatments (p=0.69).

This example shows that administration of intact whey proteins is not associated with a negative effect with respect to the trigyceride level in blood.

### EXAMPLE 2

Composition for Use in the Present Invention

An enteral composition containing whey protein with an energy density of 4.6 KJ/ml and 9% (p/v) proteins was prepared from sweet whey protein isolate. 500 g sweet whey protein isolate, 250 g maltodextrin, 20 g non-caloric sweetener, 20.3 g tri-K citrate H2O, 9.2 g MgCl2.6H2O and 5.8 g NaCl were dispersed in 4.7 Kg of demineralised water at a temperature of about 50-55° C. The pH was adjusted to 6.8 after which 300 g fatty phase were introduced the total weight of the dispersion being 5 Kg. The dispersion was homogenised and sterilised. The resulting composition had an agreeable sweet taste.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications will be covered by the appended claims.

1. Use of a composition comprising intact whey proteins for the preparation of a nutritional and/or a pharmaceutical composition for treating, preventing and/or improving metabolic dysfunctions and conditions associated with Type 2 diabetes mellitus.

2. The use according to claim 1, wherein the whey protein is sweet whey protein.

3. The use according to claim 1 or 2, wherein the amount of protein in the composition is in the range of from 1 to 90% by weight, preferably from 5 to 70% by weight, more preferably 11 to 50% by weight, even more preferably 21 to 40% by weight, most preferred about 25 to 35% by weight, based on the total dry weight of the composition.

4. The use according to any preceding claim, for enhancing post-prandial insulinemia, stimulating insulin production, and/or decreasing blood glucose levels.

5. A method for treating, preventing and/or improving metabolic dysfunctions or conditions associated with Type 2 diabetes mellitus which comprises administering an effective amount of a composition containing intact whey proteins.

6. The method of claim 6 in which intact whey proteins are administered in an amount of 0.1 to 0.8 g intact whey proteins per kg body weight.
